(19) United States
(12) Patent Application Publication
(10) Pub. No.: US 2005/0004500 A1
(43) Pub. Date: Jan. 6, 2005

(54) Device for the prevention or treatment of ulcers

(76) Inventors: James Rosser, Rye Brook, NY (US); Guadalupe Scott, Roswell, NM (US); George L. Scott III, Roswell, NM (US)

Correspondence Address: Loren G. Helmreich;Browning Bushman, P.C., Suite 1800, 5718 Westheimer, Houston, TX, 77057, US

(21) Appl. No.: 10/861,701

(22) Filed: Jun. 4, 2004

Related U.S. Application Data
(60) Provisional application No. 60/476,375, filed on Jun. 6, 2003.

(51) Int. Cl. A61F 13/00

(52) U.S. Cl. 602/41

(57) ABSTRACT

A device for the prevention or treatment of ulcers includes an absorbent layer 20 and an upper layer 30, 84 which may be transparent or opaque, and is preferably curved downward to engage the absorbent layer and minimize contact to the ulcer. A sensor or signaling member is provided for outputting an electrical signal in response to the flexible deformation of the upper layer indicative of the excessive pressure. A warning member, such as a light or alarm, is responsive to the signaling member for notifying the patient or caregiver of excessive pressure. In another embodiment, the device includes a plurality of shear detector sensors for outputting an electrical signal in response to shear movement between the absorbent layer and the patient's skin.

### FIELD OF THE INVENTION

The present invention relates to therapeutic devices used for the prevention and healing of ulcers. More particularly, the device reduces or prevents pressure application to the ulcerated regions of the patient's body, facilitates healing, and reduces the likelihood of progression of a decubitus ulcer.

### BACKGROUND OF THE INVENTION

Non-ambulatory or partially immobile people confined to beds, chairs, or wheelchairs may suffer from the formation of decubitus ulcers, also known as pressure ulcers, pressure sores, and bedsores. Decubitus ulcers are generally formed by a reduction or absence of capillary blood flow in the patient's skin for an extended period of time, which primarily results from a patient's weight-bearing bony protrusions compressing his or her skin against a hard supporting surface, such as a bed, cushion, or wheelchair, with the person remaining in the same position for more than several hours without pressure relief. The compression of the skin by the weight-bearing bony protrusion results initially in a reddening of the skin, and subsequently progresses in more advanced stages towards tissue necrosis and the formation of decubitus ulcers that may ultimately result in death, if not properly treated. The formation of a decubitus ulcer may be exacerbated by the existence of moisture from perspiration or incontinence, which is typically associated with a non-ambulatory or partially immobile person. Common problem areas for formation of decubitus ulcers typically include the heels, ankles, shoulder blades, elbows and wrists, coccyx, and ischials, which are where weight-bearing bony protrusions with adjacent areas of skin are located. Improved protective-therapeutic devices are needed for relieving pressure to the patient's skin and to therapeutically treat ulcers that have formed. In addition, a device would ideally prevent or minimize the occurrence of ulcers in high-risk patients.

U.S. Pat. No. 6,384,294 discloses a protective bandage with a compressive cushioning member. U.S. Pat. No. 6,445,304 discloses a medical alarm system for detecting excessive bleeding of patients. U.S. Pat. No. 4,667,666 discloses a bandaging device with a flat or dome shaped upper surface. The bandaging device is securable to the body about the wound. U.S. Pat. No. 6,458,109 discloses a wound treatment device with a fluid supply conduit and a fluid drainage conduit for fluid communication with the cavity.

The disadvantages of the prior art are overcome by the present invention, and an improved device and method are hereinafter provided for the prevention or treatment of ulcers.

### SUMMARY OF THE INVENTION

A device for the prevention or treatment of ulcers in one embodiment includes an absorbent layer for positioning over the ulcer and having a window therein, and a flexible transparent layer for positioning over the absorbent layer to cover the window. A signaling member outputs an electrical signal in response to the flexible deformation of the transparent layer indicative of excessive pressure on the transparent layer. A warning device responsive to the signaling member notifies the patient or caregiver of excessive pressure on the transparent layer. The transparent layer may be curved downward to engage the absorbent layer and minimize contact pressure to the ulcer.

In another embodiment, an opaque layer may be used rather than a transparent layer. The opaque layer may be hingedly connected or otherwise removably connected to the absorbent layer. The opaque layer may be selectively released from an opposing portion of the absorbent layer, thus acting as a window for viewing of the ulcer. Regardless of whether the upper layer is transparent or opaque, perforations may be provided in the upper layer, thereby allowing air movement between the ulcer and the atmosphere.

In another embodiment, the device includes an absorbent layer for positioning over the ulcer and having a window therein, an upper layer for positioning over the absorbent layer to cover the window, and a shear detector sensor for outputting an electrical signal in response to shear movement between the absorbent layer and the patient's skin. A warning member responds to the shear detector member, and notifies the patient or the caregiver of movement of the absorbent layer. A plurality of sensors may each be mounted on a tape layer secured to the patient's skin and the absorbent layer.

These and further features and advantages of the present invention will become apparent in the following detailed description, wherein reference is made to the figures in the accompanying drawings.

### BRIEF DESCRIPTION OF THE DRAWINGS

FIG. 1 is an exploded view of a suitable device according to the present invention.

FIG. 2 is a bottom view of the device shown in FIG. 1.

FIG. 3 is a top view of the device shown in FIG. 1.

FIG. 4 is a cross sectional view of another embodiment.

FIG. 5 is a top view of yet another embodiment.

FIG. 6 is a side view of a device with a hinged upper layer.

### DETAILED DESCRIPTION OF PREFERRED EMBODIMENTS

As shown in FIG. 1, a suitable inventive device includes a hydrogel membrane basal layer 15 is positioned to cover the patient's ulcer U. The transparent hydrogel layer 15 may have an oval, square or round geometry, and therapeutically assists in healing the ulcer. Over this basal layer 15 is attached on absorbent layer 20 with a center hole 25 to provide a window for viewing. The layer 20 may be formed from absorbent foam, and mounts to the patient's skin and over the hydrogel layer 15 via a suitable adhesive, such as tape. A transparent, flexible layer 30 is mounted on top of the foam membrane 20, and is positioned to cover the window 25. The layer 30 facilitates viewing of the ulcer and protects the wound from exposure to pressure or fluids. Layer 30 may thus seal with the basal layer 15, which in turn seals with the patient's skin. The member 30 may have concavity in a downward direction to further minimize or prevent contact pressure to the ulcerated area, and may be connected to the underlying foam layer 20 by an overlying layer of tape 40, which covers the outer edge of the layer 20 but does not cover or block the center hole 25 for viewing of ulcer.

Attached on the top of the transparent layer 30 is a clear plastic strip 35, which may be connected to a contact pressure alarm unit 50 in a manner to prevent the alarm from activating, except when excessive pressure is placed upon the clear layer 30, resulting in flexible deformation and displacement of the plastic strip 35, thus completing the alarm unit 50 and resulting in emission of auditory and visual alarms 55, 60. When excessive pressure is exerted on the transparent layer 30, flexible deformation and the displacement of the adjacent plastic strip 35 causes tripping of the alarm unit 50 to notify the patient and caregivers. The strip 35 may also be transparent so as not to interface with viewing the ulcer. The strip 35 may be connected at one end to the layer 30, and may extend to the unit 50, which is fixed relative to layer 30.

The alarm unit 50 may employ a timer-delay mechanism 65 to activate the warning device 55, 60 after passage of a selected time period, e.g., 2 hours or less. The warning devices may also be wired remotely to be activated at a location removed from the patient, either via wired circuitry or by a wireless communication system.

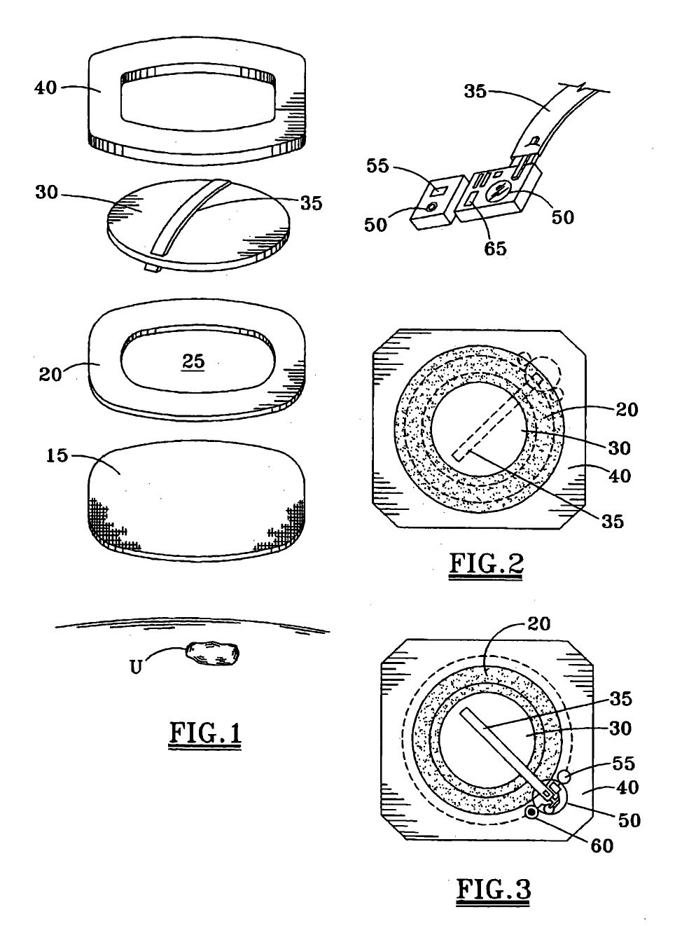

FIG. 2 is a bottom view of the device shown in FIG. 1, with the device 50 provided on the top of tape layer 40, as shown in FIG. 3.

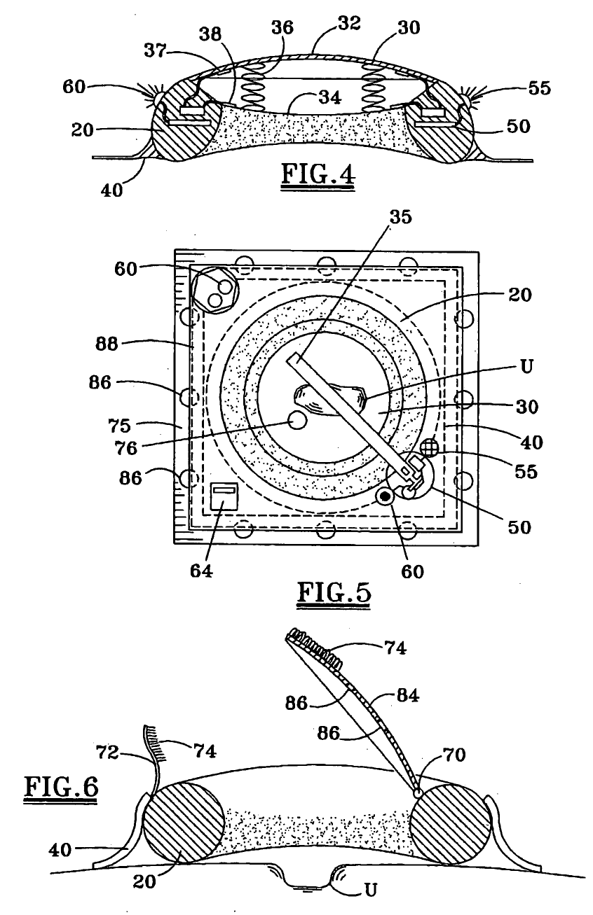

FIG. 4 illustrates an alternate embodiment of the treatment device, with a relatively thick absorbent foam layer 20, and a downwardly directed viewing layer 30, which may be coated with a thin film 32 thereon to increase light deflection and reduce heat build up proximal to the wound. Upwardly directed transparent layer 34 is positioned beneath the layer 30, and also acts to defect light which otherwise might pass through the layers 32 and 34 and to the ulcer. Springs or other biasing member 36 may be provided between the layers 30 and 34, thereby easily regulating the amount of pressure on the layer 30 to activate the alarm.

For the FIG. 4 embodiment, the alarm unit 50 is provided within the foam layer 20, and when activated may initiate the blinking of light 55, or alternatively the sending of acoustical or radio signals from transmitter 60, which signals may then be received and displayed at a nursing station.

The FIG. 4 embodiment also depicts a pair of electrical contacts 37, 38, which when engaged will complete the circuit and activate the alarm unit 50. One contact may thus be provided on the downwardly projecting layer 30, and the mating contact 38 provided on the lower upwardly directed layer 34. If layer 34 is not employed, the lower contact 38 may thus be relatively stationary on the layer 20, so that depression of the upper layer 30 a selected amount may then similarly complete the circuit and activate the alarm. Tape layer 40 secures the foam layer 20 to the patient's skin. In alternate embodiments, depression of layer 30 may pull on a strip 35 connected to layer 30, and movement of the strip may complete the circuit, as shown in FIGS. 2, 3 and 5.

In the FIG. 5 embodiment, a device includes an absorbent layer 20 which may be secured by tape 40 to underlying tape 75, which is secured to the skin of the patient. FIG. 5 illustrates an auditory and/or audio frequency transmission warning device 55 as actuated in response to excessive pressure on the upper layer, and another warning device 60 in the form of an LED blinking light. FIG. 5 also depicts a temperature sensor 64 for monitoring the patient's skin surface temperature within the area of the ulcer and thus radially within the absorbent layer, and/or for monitoring the air temperature under the layer 30, above the ulcer, and radially within the absorbent layer. A plug 76 may be provided in the upper layer, with the plug being formed from a suitable material for receiving a needle. Fluids may thus be inserted through the plug in an aspiration injection, or fluid may be removed from the area, e.g., for catheter drainage.

FIG. 5 also illustrates a plurality of shear sensors 86 which are attached to the tape layer 75 and to the absorbent layer 40. A suitable conductor such as wire 88 may be used for interconnecting each of the sensors with shear detector alarm unit 80. Shear detector alarm unit 80 similarly may include warning devices 55 and 60.

Upon shear movement between the absorbent layer 40 and the tape layer 75, and thus between the absorbent layer 40 and the patient's skin, one of the members 86 may be triggered, and a signal sent to the shear detector alarm unit 80. The selected positioning and placement of the detectors 86 will depend upon the size and configuration of the device.

FIG. 6 depicts simplistically another embodiment of the device, wherein the absorbent layer 20 is shown to have a generally circular cross section. FIG. 6 also illustrates a tape layer 40 for taping the absorbent layer to the patient's skin.

The device includes an upper layer 84 which may be opaque to avoid problems with respect to effect of sunlight. In this case, the upper layer is removably connected to the absorbent layer. In the depicted embodiment, the upper layer is connected at one side to absorbent layer 20 by hinged connection 70, and a suitable latch body 72 provided on the closing side of the absorbent member. For the FIG. 6 embodiment, a latch consists of a velcro type material 74 on the end of the upper layer, and on latch body 72. In other embodiments, the upper layer could be completely removable from the absorbent layer 20. A removable connection could be made between layer 20 and upper layer 84 by a velcro type material, by tape, or by another reuseable connection material. The FIG. 6 embodiment may alternatively utilize a transparent upper layer, thereby allowing the practitioner to perform wound debridgement and other procedures by removing the transparent layer. For each of the embodiments, the top layer, whether a transparent layer 30 or an opaque layer 84, may be provided with one or more perforations 86 for limited communication of air between the ulcer and the atmosphere.

The foregoing disclosure and description of the invention is illustrative and explanatory of preferred embodiments. It would be appreciated by those skilled in the art that various changes in the size, shape of materials, as well as in the details of the illustrated construction or combination of features discussed herein may be made without departing from the spirit of the invention, which is defined by the following claims.

1. A device for the prevention or treatment of ulcers, comprising:
an absorbent layer for positioning over the ulcer and having a window therein;
a flexible transparent layer for positioning over the absorbent layer to cover the window;
a signaling member for outputting an electrical signal in response to the flexible deformation of the transparent layer indicative of excessive pressure on the transparent layer; and
a warning member responsive to the signaling member for notifying the patient or caregiver of excessive pressure on the transparent layer.

2. A device as defined in claim 1, wherein the transparent layer is curved downward to engage the absorbent layer and minimize contact pressure to the ulcer area.

3. A device as defined in claim 1, wherein the signaling member is a form of an elongate strip mounted at one end to the transparent layer.

4. A device as defined in claim 3, wherein the signaling member is transparent.

5. A device as defined in claim 1, wherein the signaling member includes a contact member connected to the transparent layer for electrical contact with a mating contact member of the signaling member to output the electrical signal.

6. A device as defined in claim 1, wherein the transparent layer includes one or more through apertures.

7. A device as defined in claim 1, further comprising:
a delay mechanism for activating the warning member in response to the signaling member after a preselected period of time.

8. A device as defined in claim 1, wherein the absorbent layer is mounted to a hydrogel membrane layer positioned over the ulcer.

9. A device as defined in claim 1, wherein the absorbent layer is a foam layer.

10. A device as defined in claim 1, wherein the transparent layer is connected to the absorbent foam layer by tape which covers an outer edge of the absorbent layer.

11. A device as defined in claim 1, further comprising:
one or more other signaling members for outputting another electrical signal in response to shear movement of the absorbent layer relative to the ulcer.

12. A device as defined in claim 1, further comprising:
an alarm unit positioned within the absorbent layer, the alarm unit receiving signals from the signaling member and outputting signals to the warning member.

13. A device as defined in claim 1, wherein the warning member is positioned at least in part within the absorbent layer.

14. A device as defined in claim 1, further comprising:
a second transparent layer positioned below the transparent layer and above the ulcer, the second transparent layer being curved upward to deflect light away from the ulcer.

15. A device as defined in claim 14, further comprising:
the signaling member is electrically connected to electrical contacts mounted on the transparent layer and the second transparent layer.

16. A device for the treatment of ulcers as defined in claim 14, further comprising:
a biasing member for biasing the transparent layer from the second transparent layer.

17. A device as defined in claim 1, further comprising:
a film on the transparent layer for deflecting light from the ulcer.

18. A device as defined in claim 1, further comprising:
a temperature sensor for measurement of one of air temperature and skin surface temperature radially within the absorbent layer.

19. A device for the prevention or treatment of ulcers, comprising:
an absorbent layer for positioning over the ulcer and having a window therein;
a flexible layer for positioning over the absorbent layer to cover the window;
a removable connection between the flexible layer and the absorbent layer for moving the flexible layer to view the ulcer;
a signaling member for outputting electrical signals in response to flexible deformation of the flexible layer indicative of excessive pressure on the flexible layer; and
a warning member responsive to the signaling member for notifying the patient or caregiver of excessive pressure on the flexible layer.

20. A device as defined in claim 19, wherein the flexible layer includes one or more perforations for communication between the ulcer radially within the absorbent layer and atmosphere.

21. A device as defined in claim 19, wherein the flexible layer is curved downward to engage the absorbent layer and minimize contact to the ulcer.

22. A device as defined in claim 19, further comprising:
one or more other signaling members for outputting another electrical signal in response to shear movement of the absorbent layer relative to the ulcer.

23. A device as defined in claim 19, further comprising:
the signaling member includes an electrical contact mounted on the flexible layer, such that flexible deformation of the flexible layer makes electrical contact with a mating contact member to output the electrical signal.

24. A device as defined in claim 19, wherein the removable connection includes a hinged connection between the flexible layer and the absorbent layer, and a latch member for removably latching the flexible layer to the absorbent layer.

25. A device as defined in claim 19, wherein the flexible layer is an opaque layer.

26. A device for the prevention or treatment of ulcers, comprising:
an absorbent layer for positioning over the ulcer and having a window therein;
an upper layer for positioning over the absorbent layer to cover the window;
a shear detector sensor for outputting an electrical signal in response to shear movement between the absorbent layer and the patient's skin; and
a warning member responsive to the shear detector sensor for notifying the patient or caregiver of movement of the absorbent layer.

27. A device as defined in claim 26, wherein the shear detector sensor includes a plurality of sensors each mounted between a tape layer secured to the patient's skin and the absorbent layer.
